(19) United States
(12) Patent Application Publication
(10) Pub. No.: US 2025/0218579 A1
(43) Pub. Date: Jul. 3, 2025

(54) SYSTEMS, METHODS AND INTERFACES FOR RESOURCE RESERVATION USING SMART CONTRACTS

(71) Applicant: Elevance Health, Inc., Indianapolis, IN (US)

(72) Inventors: Atul APTE, Indianapolis, IN (US); Akshay BHAKRI, Indianapolis, IN (US); Venkata Subramanian Muralisrinivasan, Bengaluru (IN); Jeeva S V, Bengaluru (IN); Elayabharathi Elayaperumal, Hosur (IN)

(73) Assignee: Elevance Health, Inc., Indianapolis, IN (US)

(21) Appl. No.: 18/986,676

(22) Filed: Dec. 18, 2024

Related U.S. Application Data
(60) Provisional application No. 63/615,361, filed on Dec. 28, 2023.

(51) Int. Cl. G16H 40/20; G06Q 10/1093; G16H 10/60

(52) U.S. Cl. CPC G16H 40/20; G06Q 10/1095; G16H 10/60; G06Q 2220/10

(57) ABSTRACT

A system and method are provided for resource reservation using smart contracts. The method may be performed at a permissioned blockchain network coupled to clients. The permissioned blockchain network may include an ordering service and a plurality of members. Each member may include (i) an endorsing peer node for endorsing transactions and (ii) a committing peer node for validating and committing transactions. The method may include, at each member (i) hosting a respective ledger, (ii) receiving, from the clients, using application programmers interfaces (APIs), via graphical user interfaces, a proposal for reserving one or more resources, (iii) in response to receiving the reservation proposal, (a) executing a smart contract on the respective ledger, to initiate a transaction for reserving the one or more resources, and (b) indicating, to the client, a successful reservation or a rejection of the reservation of the one or more resources, via the graphical user interfaces.

### RELATED APPLICATIONS

This application claims priority to U.S. Provisional Application Ser. No. 63/615,361, filed Dec. 28, 2023, entitled “System and Method for Appointment Booking Using Blockchain,” which is incorporated by reference herein in its entirety.

### TECHNICAL FIELD

The invention relates to systems, methods and interfaces for resource reservation, and is more particularly, but not by way of limitation, directed to technology for resource reservation using smart contracts.

### BACKGROUND

Resource reservation systems can enhance the utilization of staff, facilities, computing, storage and network resources while reducing wait times. In healthcare, for example, appointment booking can enhance the utilization of medical resources, including online systems. Multiple entities may simultaneously require assistance with resource reservations. With conventional systems, simultaneous access of the resource reservation system may lead to bottlenecks and delays. Furthermore, there may be several factors to take into account for resource reservation, including service level agreements, reducing costs, increasing satisfaction, reducing waiting time, conflict resolution and improving fairness. In some domains, such as in healthcare, there are also issues with protecting sensitive information while reserving resources.

### SUMMARY

Accordingly, there is a need for tools, systems, methods and interfaces for resource reservation. Some embodiments use smart contracts to facilitate resource reservation whereby only parties that are part of decision making are notified or informed about a reservation. Third party (e.g., a caregiver) may act on behalf of a party (e.g., a patient) requiring a reservation. Personal or protected information (e.g., protected health information or PHI, in the case of healthcare) may be shielded from third party. At the same time, different entities may continue to have up-to-date information regarding available resources and resource reservations. The resource reservation may be facilitated via graphical user interfaces that are specialized for each entity. Some embodiments provide application programmers interfaces (APIs) to interface with a blockchain network and components therein, and/or the graphical user interfaces, making it possible to implement complex resource reservation systems. Some embodiments provide a blockchain-enabled appointment booking system to enable consumers, such as patients, caregivers, payers, and/or providers to function as transactional peers and participate in appointment management transactions.

One or more embodiments of the invention are directed to an improved system and method for resource reservation using smart contracts and/or smart interfaces. The method may be performed at a system or a server having one or more processors, memory, one or more displays, and one or more programs stored in the memory and configured for execution by the one or more processors, the one or more programs including instructions for performing the steps described herein.

The system may include a permissioned blockchain network coupled to one or more client devices. The permissioned blockchain network may include an ordering service with three orderer nodes and at least three members. Each member may include (i) an endorsing peer node configured to endorse transactions and (ii) a committing peer node to validate and commit transactions. Each member may be configured to (i) host a respective ledger, (ii) receive, from a client device of the one or more client devices, a proposal for reserving one or more resources, (iii) in response to receiving the reservation proposal, execute a smart contract of a plurality of smart contracts on the respective ledger, to initiate a transaction for reserving the one or more resources. The system may also include a user interface component coupled to the permissioned blockchain network. The user interface component may be configured to provide a plurality of application programmers interfaces (APIs) to access a plurality of graphical user interfaces.

In some embodiments, the at least three members includes patients, providers, and caregivers.

In some embodiments, the plurality of smart contracts includes an appointment contract, a patient contract, a provider contract and a caregiver contract.

In some embodiments, the appointment contract is configured to perform one or more operations selected from the group consisting of: creating an appointment initiated by one or more patients, one or more providers, or one or more caregivers; listing an appointment in response to queries for appointments based on a patient identifier from the respective ledger; updating one or more appointments with respect to the one or more patients, the one or more providers, or the one or more caregivers; referring appointments to let a provider refer an appointment to another provider; and querying all the appointments based on a query pattern.

In some embodiments, the patient contract may be configured to perform one or more operations selected from the group consisting of: enrolling a new patient into the permissioned blockchain network; querying patient information in the respective ledger, in response to a query from one or more patients, one or more providers, or one or more caregivers; and updating patient information in response to a request from the one or more patients.

In some embodiments, the provider contract may be configured to perform one or more operations selected from the group consisting of: enrolling a new provider into the permissioned blockchain network; querying provider information in the respective ledger, in response to a query from one or more patients, one or more providers, or one or more caregivers; and updating provider information in response to a request from the one or more providers.

In some embodiments, the caregiver contract may be configured to perform operations selected from the group consisting of: enrolling a new caregiver into the permissioned blockchain network; querying caregiver information in the respective ledger, in response to a query from one or more patients, one or more providers, or one or more caregivers; updating caregiver information in response to a request from the one or more caregivers; and controlling appointment of a patient's appointment.

In some embodiments, the plurality of graphical user interfaces may include one or more graphical user interfaces for client login, client signup, a plurality of dashboards, each dashboard corresponding to a respective member type, an interface for a calendar view, an interface for booking resources, an interface for accepting resource reservation, an interface for rejecting resource booking, an interface for client profile management, an interface for displaying current users.

In some embodiments, the plurality of graphical user interfaces may include a corresponding graphical user interface for patients, a corresponding interface for providers, and a corresponding interface for caregivers.

In some embodiments, the plurality of graphical user interfaces may include an interface with one or more affordances for providers to refer an appointment to another provider.

In some embodiments, the permissioned blockchain network may be configured to, at a member of the at least three members, receive a connection request from a client device of the one or more client devices. The permissioned blockchain network may also be configured to, at a smart contract: process the connection request to select (i) a graphical user interface of the plurality of graphical user interfaces based on the client device and (ii) an API corresponding to the graphical user interface; provide, via the API, the graphical user interface, to the client device; receive, via the graphical user interface, a selection of a resource of a plurality of resources, from the client device; and in response to the selection of the resource, perform, using the plurality of graphical user interfaces and/or the respective ledger, one or more operations selected from the group consisting of: client login, client signup, and display (i) one or more dashboards, each dashboard corresponding to a respective member type, (ii) an interface for a calendar view, (ii) an interface for booking resources, (iii) an interface for accepting resource reservation, (iv) an interface for rejecting resource booking, (v) an interface for client profile management, or (vi) an interface for displaying current users.

In another aspect, a method may be provided for resource reservation using smart contracts and/or smart interfaces. The method may be performed at a permissioned blockchain network coupled to one or more client devices. The permissioned blockchain network may include an ordering service with three orderer nodes and at least three members. Each member may include (i) an endorsing peer node for endorsing transactions and (ii) a committing peer node for validating and committing transactions. The method may include, at each member (i) hosting a respective ledger, (ii) receiving, from the one or more client devices, using one or more application programmers interfaces (APIs), via one or more graphical user interfaces of a plurality of graphical user interfaces, a proposal for reserving one or more resources, (iii) in response to receiving the reservation proposal, (a) executing a smart contract of a plurality of smart contracts on the respective ledger, to initiate a transaction for reserving the one or more resources, and (b) indicating, to the client device, a successful reservation or a rejection of the reservation of one or more resources, via the one or more graphical user interfaces.

In some embodiments, the at least three members may include patients, providers, and caregivers.

In some embodiments, the plurality of smart contracts may include an appointment contract, a patient contract, a provider contract and a caregiver contract.

In some embodiments, the appointment contract may include operations selected from the group consisting of: creating an appointment initiated by one or more patients, one or more providers, or one or more caregivers; listing an appointment in response to queries for appointments based on a patient identifier from the respective ledger; updating one or more appointments with respect to one or more patients, one or more providers, or one or more caregivers; referring appointments to let a provider refer an appointment to another provider; and querying all the appointments based on a query pattern.

In some embodiments, the appointment contract may include operations selected from the group consisting of: enrolling a new patient into the permissioned blockchain network; querying patient information in the respective ledger, in response to a query from one or more patients, one or more providers, or one or more caregivers; and updating patient information in response to a request from the one or more patients.

In some embodiments, the provider contract may include operations selected from the group consisting of: enrolling a new provider into the permissioned blockchain network; querying provider information in the from the respective ledger, in response to a query from one or more patients, one or more providers, or one or more caregivers; and updating provider information in response to a request from the one or more providers.

In some embodiments, the caregiver contract may include operations selected from the group consisting of: enrolling a new caregiver into the permissioned blockchain network; querying caregiver information in the from the respective ledger, in response to a query from one or more patients, one or more providers, or one or more caregivers; updating caregiver information in response to a request from the one or more caregivers; and controlling appointment of a patient's appointment.

In some embodiments, the plurality of graphical user interfaces may include one or more graphical user interfaces for client login, client signup, a plurality of dashboards, each dashboard corresponding to a respective member type, an interface for a calendar view, an interface for booking resources, an interface for accepting resource reservation, an interface for rejecting resource booking, an interface for client profile management, an interface for displaying current users.

In some embodiments, the plurality of graphical user interfaces may include a corresponding graphical user interface for patients, a corresponding interface for providers, and a corresponding interface for caregivers.

In some embodiments, the plurality of graphical user interfaces may include an interface with one or more affordances for providers to refer an appointment to another provider.

In some embodiments, the method includes, at a member of the at least three members, receiving a connection request from a client device of the one or more client devices. The method may also include, at a smart contract: processing the connection request to select (i) a graphical user interface of the plurality of graphical user interfaces based on the client device and (ii) an API corresponding to the graphical user interface; providing, via the API, the graphical user interface, to the client device; receiving, via the graphical user interface, a selection of a resource of a plurality of resources, from the client device; and in response to the selection of the resource, performing, using the plurality of graphical user interfaces and/or the respective ledger, one or more operations selected from the group consisting of: client login, client signup, and display (i) one or more dashboards, each dashboard corresponding to a respective member type, (ii) an interface for a calendar view, (ii) an interface for booking resources, (iii) an interface for accepting resource reservation, (iv) an interface for rejecting resource booking, (v) an interface for client profile management, or (vi) an interface for displaying current users.

In some embodiments, a computer system has one or more processors, memory, and a display. The one or more programs include instructions for performing any of the methods described herein.

In some embodiments, a non-transitory computer readable storage medium stores one or more programs configured for execution by a computer system having one or more processors, memory, and a display. The one or more programs include instructions for performing any of the methods described herein.

### BRIEF DESCRIPTION OF THE DRAWINGS

FIG. 1 is a schematic diagram of an example system for resource reservation using smart contracts and/or smart interfaces, according to some embodiments;

FIG. 2A is an example user interface for a provider login to a resource reservation system, according to some embodiments;

FIG. 2B shows an example user interface for a caregiver login to a resource reservation system, according to some embodiments;

FIG. 2C shows an example user interface for a dashboard for a caregiver, according to some embodiments;

FIG. 2D shows an example user interface for another dashboard for a caregiver, according to some embodiments;

FIG. 2E shows an example user interface for a dashboard for a provider, according to some embodiments;

FIG. 2F shows an example user interface for a dashboard for a patient, according to some embodiments;

FIG. 2G shows an example user interface for a calendar view for a caregiver, according to some embodiments;

FIG. 2H shows an example user interface for a caregiver to schedule an appointment, according to some embodiments;

FIG. 2I shows an example user interface for a patient to accept an appointment, according to some embodiments;

FIG. 2J shows an example user interface for a patient to reject an appointment, according to some embodiments;

FIG. 2K shows an example user interface for a caregiver to create a profile, according to some embodiments;

FIG. 2L shows an example user interface for a dashboard for a caregiver, according to some embodiments;

FIG. 3A shows a flowchart of an example method for a patient to book an appointment with a provider without a caregiver, according to some embodiments;

FIG. 3B shows a flowchart of an example method for a patient to book an appointment with a provider when patient has a caregiver, according to some embodiments;

FIG. 3C shows a flowchart of an example method for a caregiver to book an appointment with a provider on behalf of a patient, according to some embodiments;

FIG. 3D shows a flowchart of an example method for a provider to book an appointment with a patient, according to some embodiments;

FIG. 3E shows a flowchart of an example method for a referral process, according to some embodiments.

### DETAILED DESCRIPTION OF THE PREFERRED EMBODIMENTS

The following descriptions of embodiments of the invention are exemplary, rather than limiting, and many variations and modifications are within the scope and spirit of the invention. Although numerous specific details are set forth in order to provide a thorough understanding of the present invention, it will be apparent to one of ordinary skill in the art, that embodiments of the invention may be practiced without these specific details. In other instances, well-known features have not been described in detail in order to avoid unnecessarily obscuring the present invention.

One or more embodiments of the invention are directed to an improved method and system for reserving resources using smart contracts and/or smart interfaces.

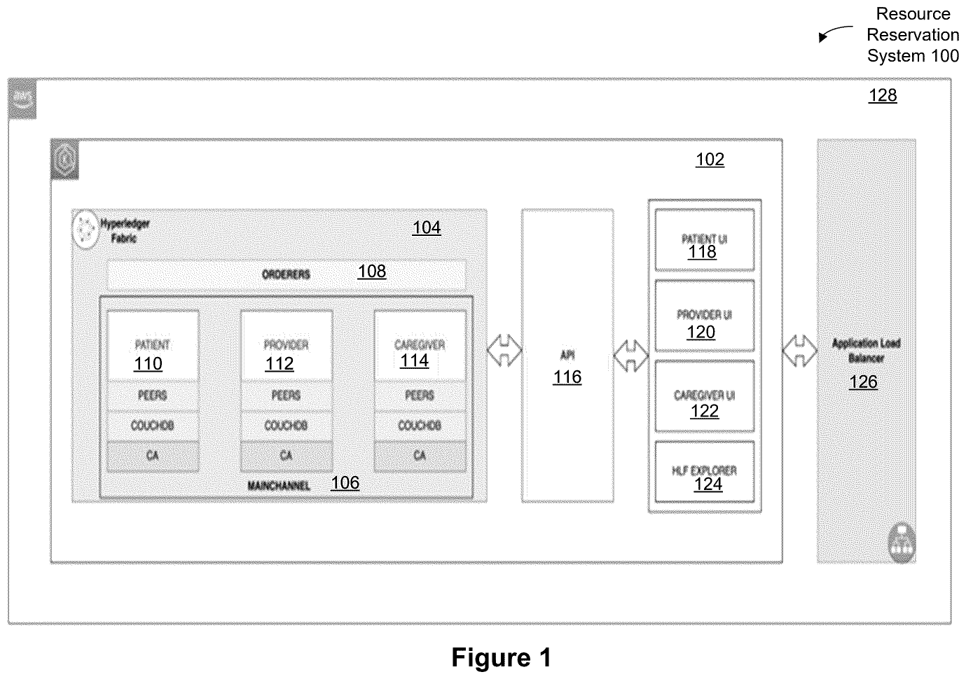

FIG. 1 is a schematic diagram of an example system 100 for resource reservation using smart contracts and/or smart interfaces, according to some embodiments. The system 100 may include a permissioned blockchain network 104 (e.g., Hyperledger Fabric) that may include orderer nodes 108, and members (sometimes referred to as organizations, e.g., a patient 110, a provider 112, and a caregiver 114), each of which may include corresponding peer nodes, a clustered database that may be used to a logical database server on any number of servers or virtual machines (VMs), and/or a certificate authority (CA). The members may be connected via one or more channels 106. A channel may be a private subnetwork for communication between the members 110, 112, and/or 114 (and more channels may be present in alternative embodiments), for the purpose of conducting private and confidential transactions. A channel may be defined by the members, anchor peers per member, a shared ledger, chaincode or smart contract applications, and/or orderers 108 (sometimes referred to as ordering service nodes). Each transaction on the network may be executed on a channel, e.g., the channel 106. Each party may be authenticated and authorized to transact on that channel. Each peer that joins a channel, may have its own identity provided by a services provider, which may authenticate each peer to its peers (for a channel) and corresponding services.

For the permissioned blockchain network, Fabric, Quorum, Corda, or private Ethereum are example permissioned blockchain networks that can add a layer of security to allow only authorized participants in the network. For consensus, Fabric follows the Practical Byzantine Fault Tolerance (PBFT) or RAFT consensus which is more fault tolerant and performance oriented when compared to other blockchain networks that use Proof-of-Stake (PoS) or Proof-of-Work (PoW) consensus and Corda that uses Consensus by Agreement. Fabric is designed to be scalable and customizable when compared to Ethereum and Corda. Some embodiments may use Fabric features, such as multiple channels, smart contracts as a service, deployment to the cloud, private transactions, event listeners, and/or customizable ledger data. Depending on the application, different permissioned blockchain frameworks may be used. For example, Fabric may be used for health care and supply chain, whereas the other blockchains may be used for financial and supply chain focused technologies.

The system 100 may include one or more application programmers interfaces (APIs) 116 to interface with the permissioned blockchain network 104 and a plurality of graphical user interfaces (UIs) (e.g., a patient UI 118, a provider UI 120, a caregiver UI 122, and/or an explorer UI 124). Each of the UIs may be specialized for a particular type of client, or may be used to show as further described below. The explorer UI may include a Hyperledger explorer that is configured with the blockchain network for providing a view of the nodes, organizations, chain codes, transactions, channels, blocks of the blockchain network.

The permissioned blockchain network 104, the APIs 116 and/or the UIs may be configured in a control plane 102 (e.g., a Kubernetes control plane) of a cloud service instance 128 (e.g., Amazon Web Service). The control plane 102 may include services that interact with a data plane. The cloud service instance 128 may be a managed service that eliminates the need to install, operate, and maintain own Kubernetes control plane. Kubernetes is an example open-source system that automates the management, scaling, and deployment of containerized applications.

The system 100 may include an application load balancer 126 that interfaces with one or more applications (sometimes referred to as client devices, clients or client applications).

Some embodiments use inclusive transaction protocols whereby any of the members or parties may act as primary entities in any transaction. The network participants may belong to a same channel. Primary entities may be the entities who initiated and are involved in that transaction. For example, (i) a patient and a provider may act as primary entities, and a caregiver, a payer, other providers may act as related entities; (ii) a caregiver and a provider may act as primary entities, and a patient, a payer, and other providers may act as related entities; (iii) two providers may act as primary entities and a patient, a caregiver, and a payer may act as related entities; (iv) a payer and a patient may act as primary entities, and a caregiver and providers may act as related entities. The patient and the caregiver nodes may be managed by a payer system (e.g., when each patient and caregiver cannot have their own private nodes).

Confirmation of resources reservations (e.g., appointments) may be driven by consensus and provenance. Shared ledger and blockchain managed appointment book ensures that directly or indirectly connected entities have access to relevant confirmed resource reservations. Digitally confirmed and/or verified transactions or appointments enhance the service level and service experience for consumers. An appointment book may be maintained as part of a shared ledger that enables organizations of a blockchain network to have a common and trusted visibility into the entire pool of appointments. Appointments may provide early insights into evolving patient-provider relationships (e.g., primary physicians, providers, caregivers), healthcare needs and patient actions. Blockchain may provide the capability of enabling and using multi-organization consensus to prevent posting of bad transactions (e.g., duplicate or overlapping transactions) in an ecosystem because more than two or a majority of the organizations have to agree on the transactions before getting posted on the blockchain ledger. Blockchain can be used to establish a source of truth when dispute resolution is an established or operational process. Blockchain ledger may provide end to end visibility and traceability of transactions on the network. Payers, community or public health organizations can gain insights by observing appointments.

Although the description uses an appointment booking system for illustrating the concepts, the techniques may be used to reserve any type of shared resources, and/or in scenarios other than healthcare.

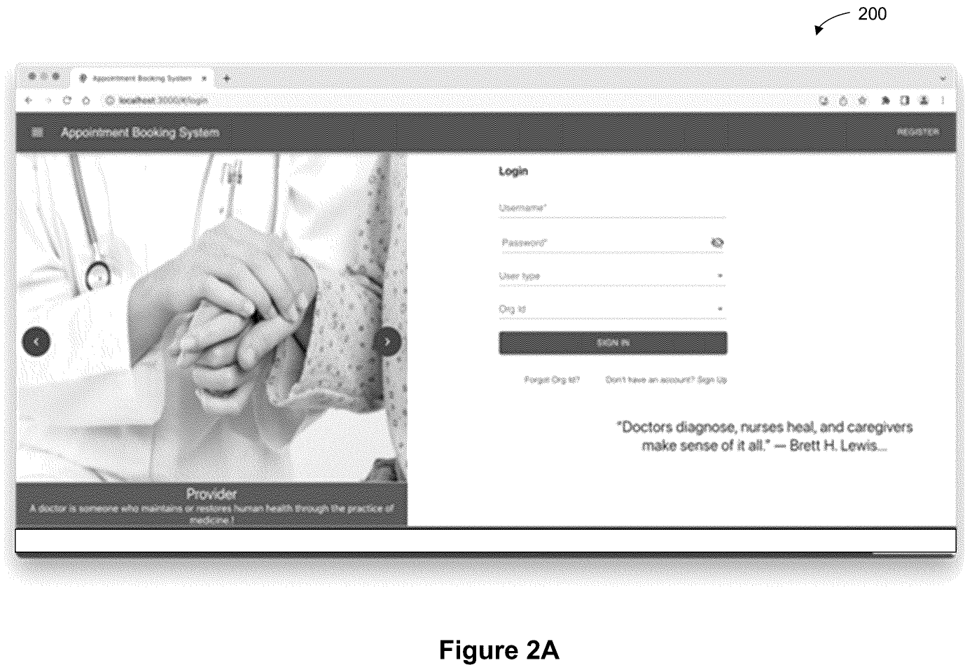

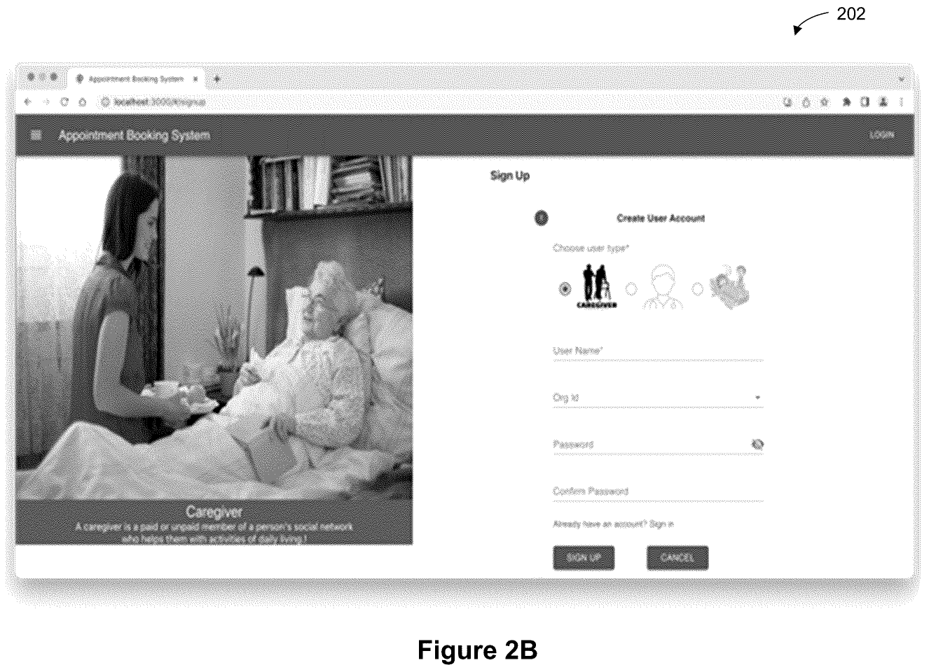

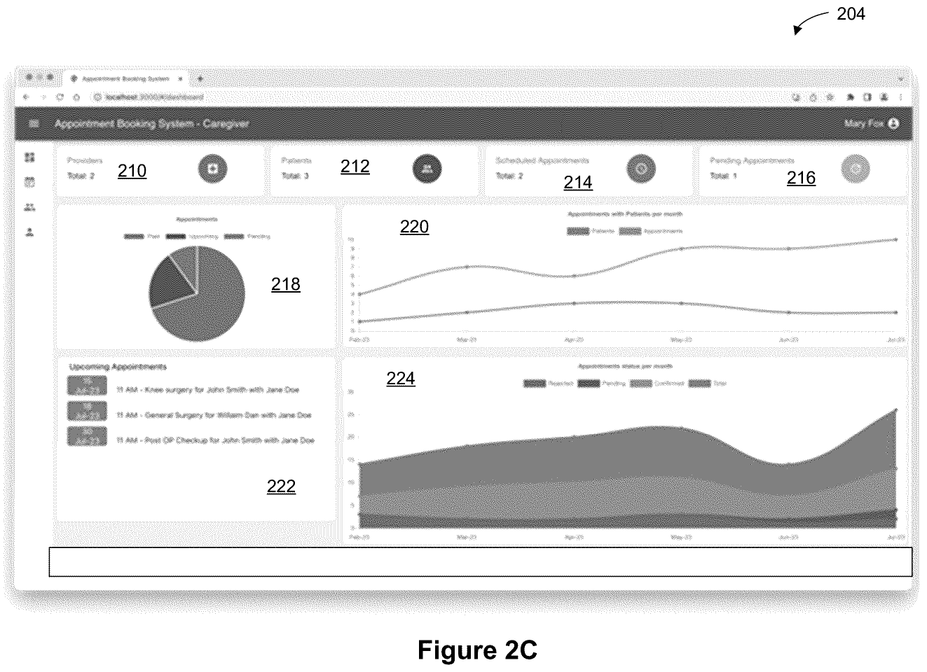

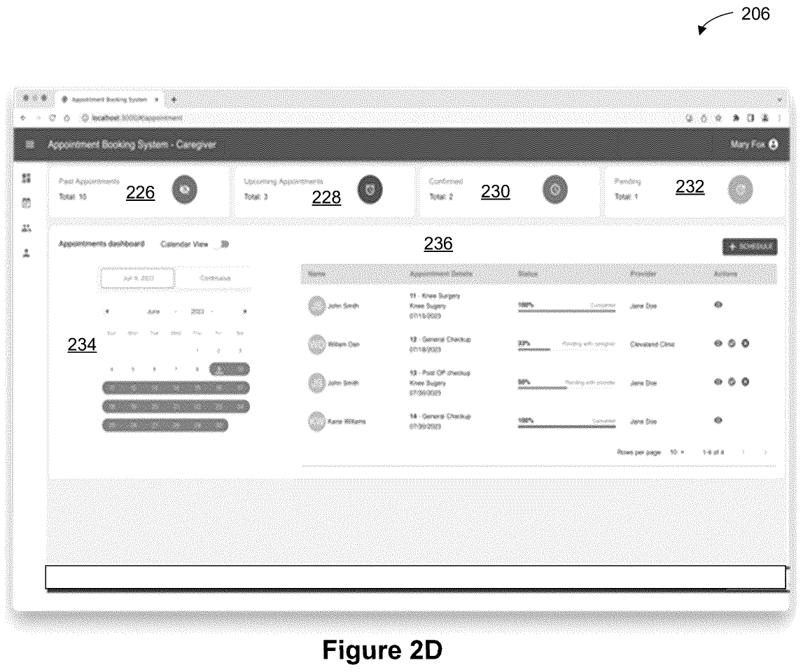

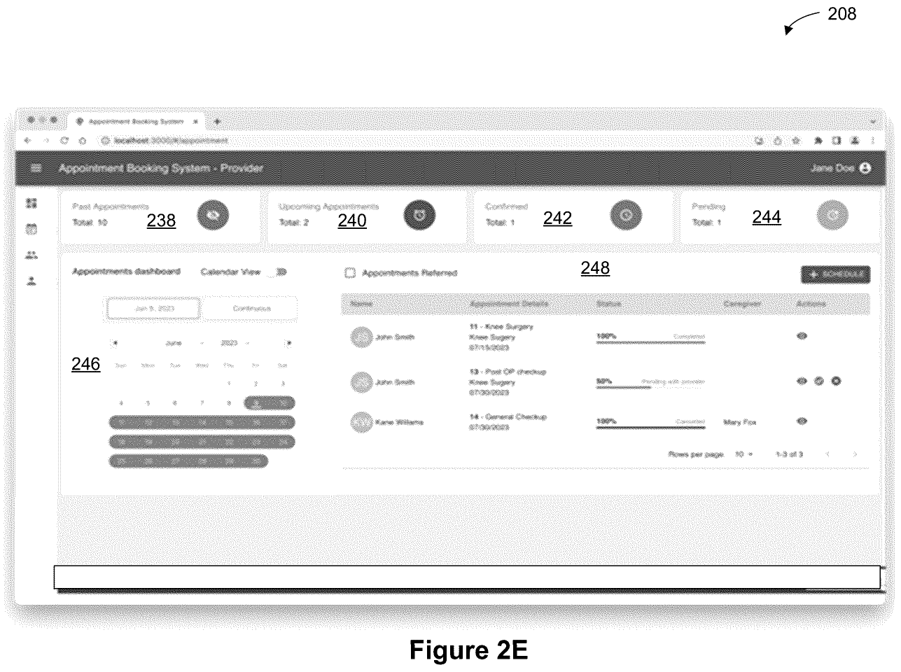

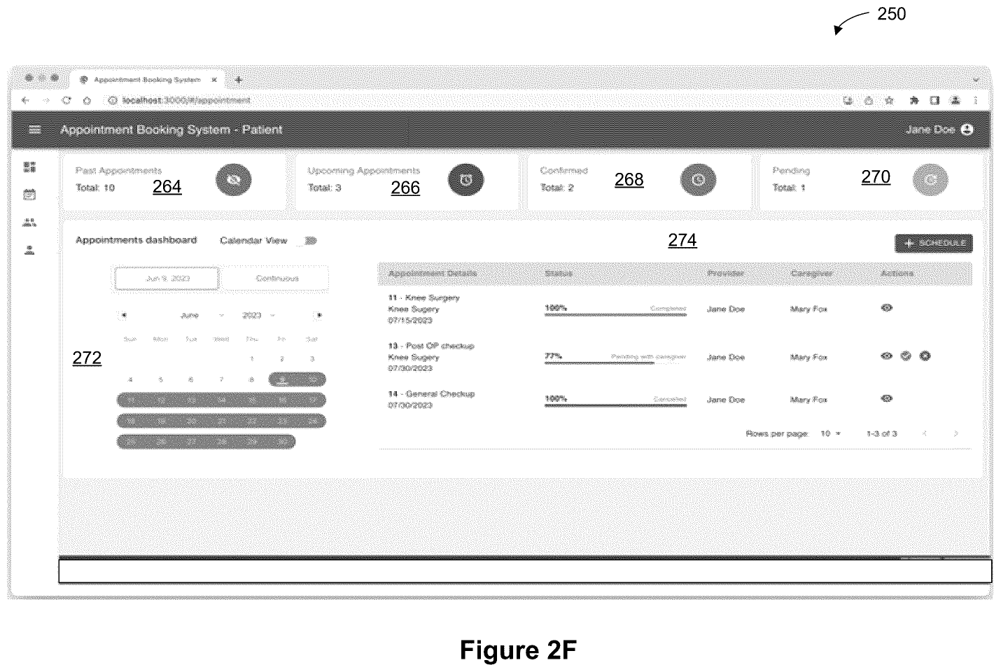

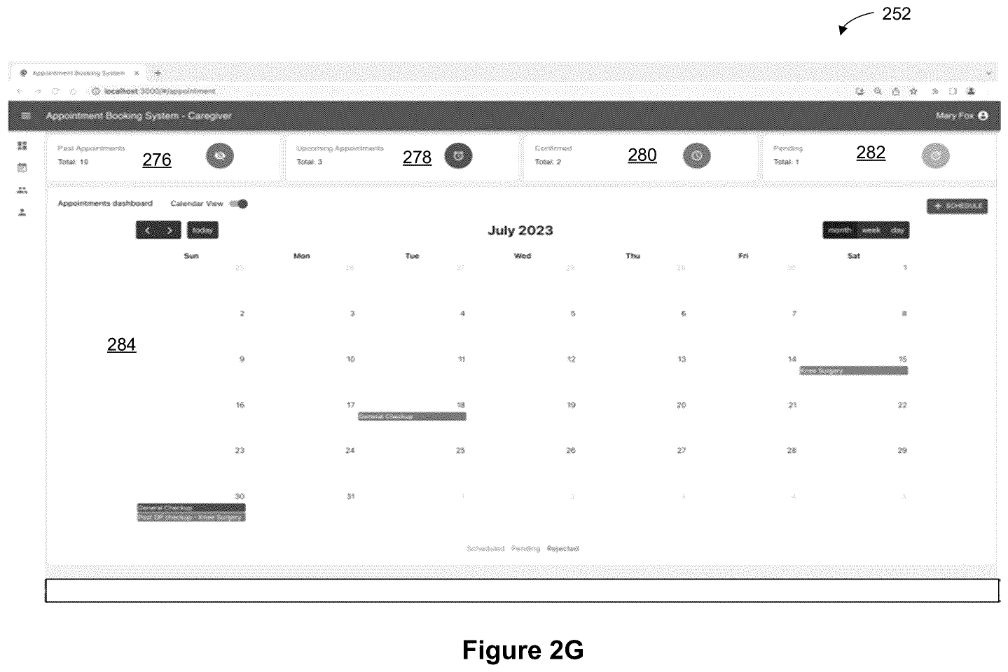

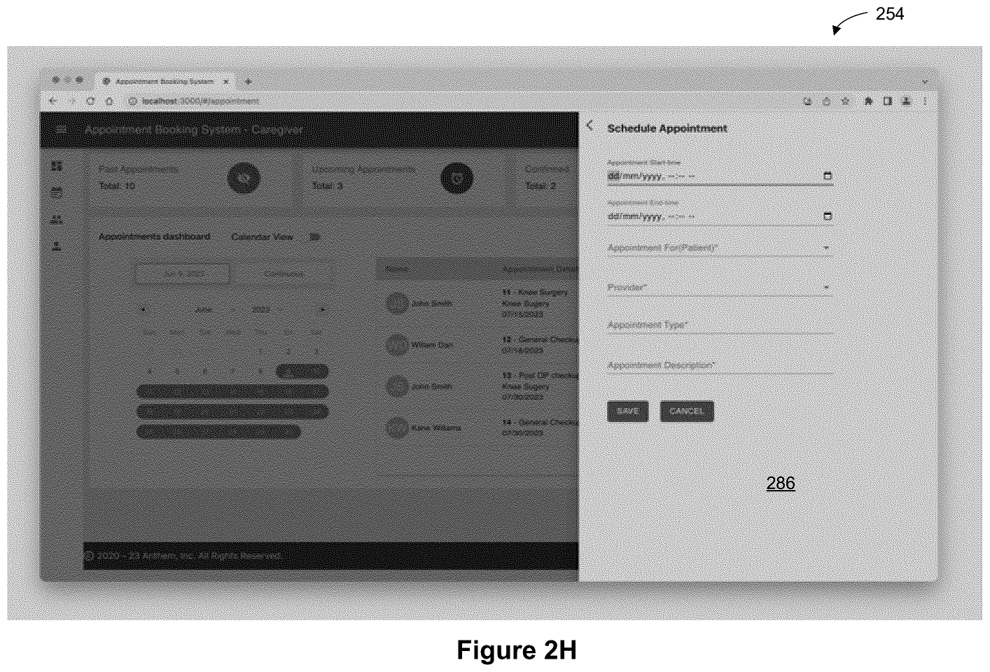

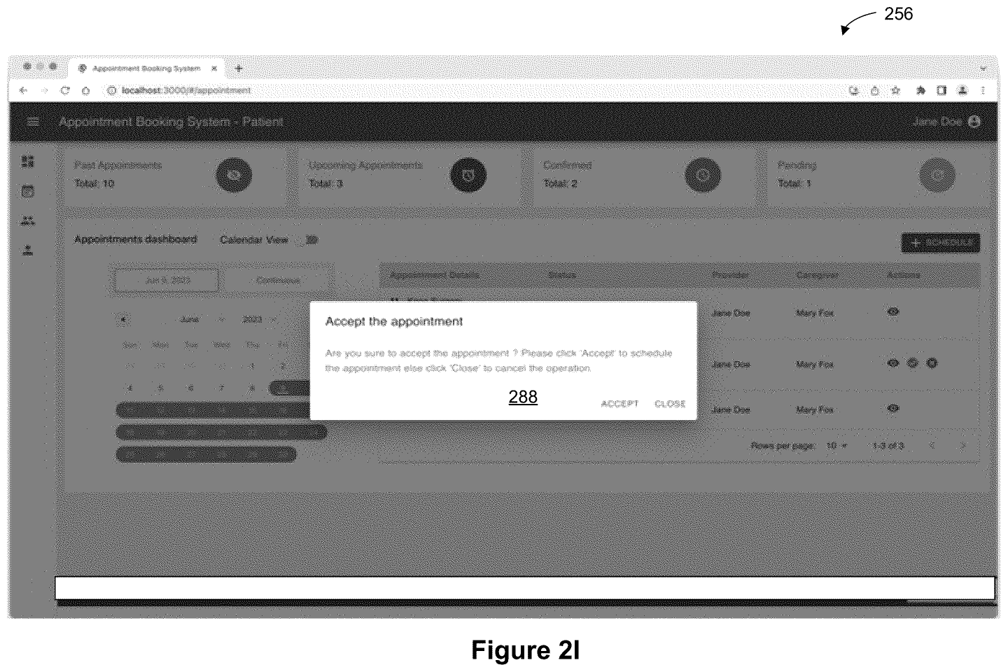

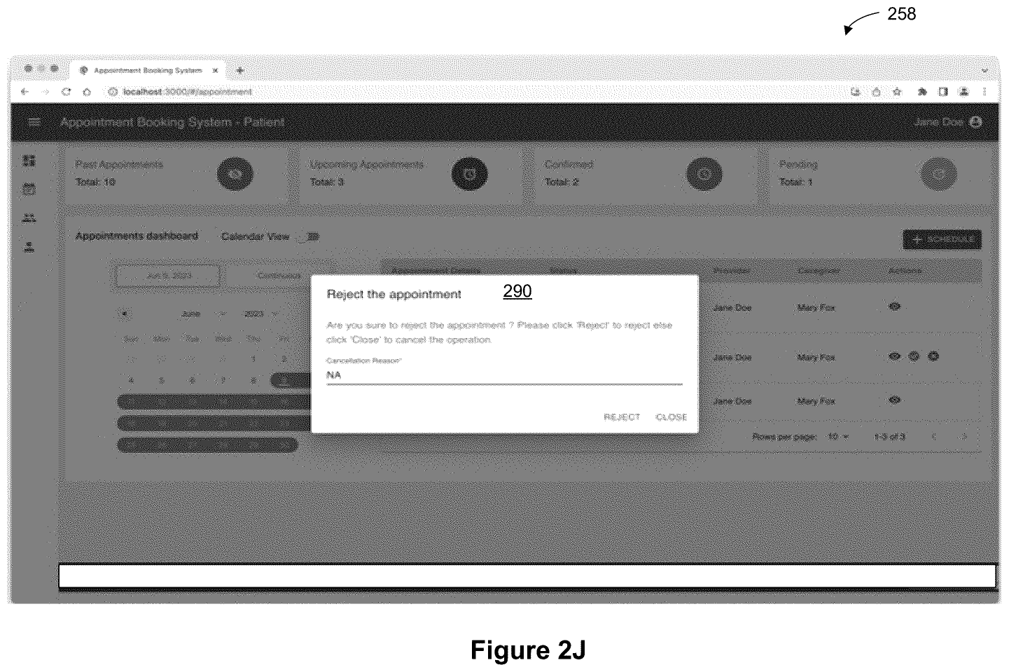

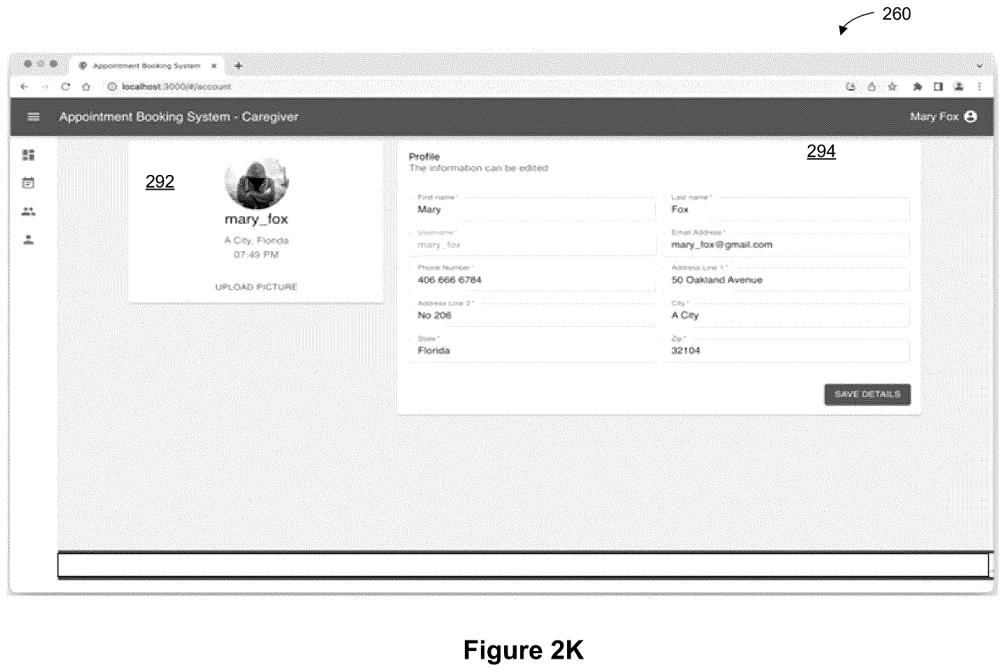

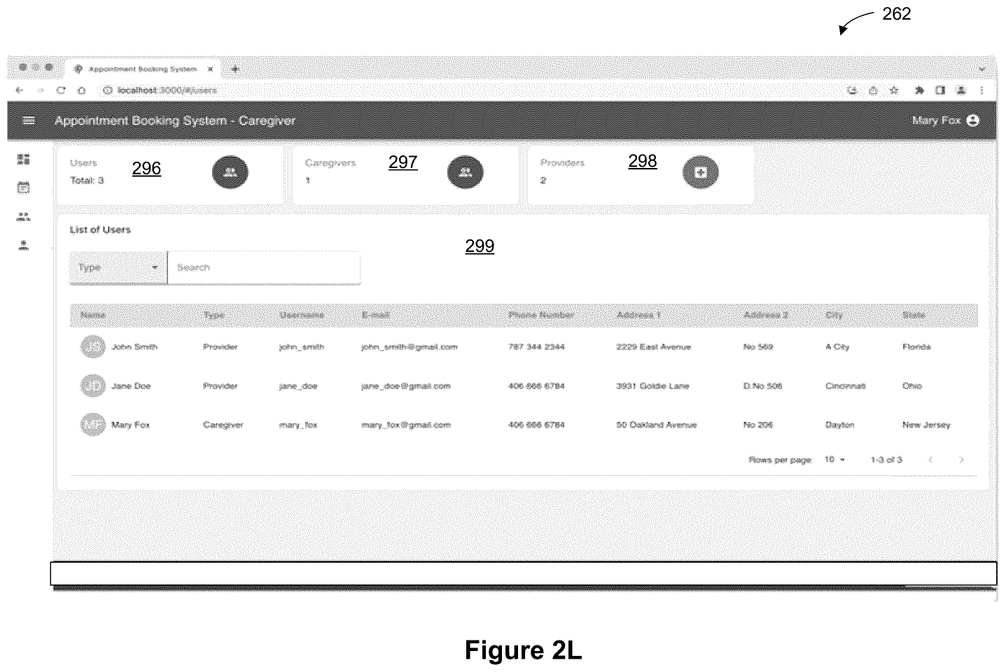

FIG. 2A is an example user interface 200 for a provider login to a resource reservation system, according to some embodiments. The interface 200 may be used by a provider (e.g., a doctor) that provides services to patients, to reserve and/or manage resources (e.g., book appointments). The provider may authenticate with the system using the interface 200.

FIG. 2B shows an example user interface 202 for a caregiver login to a resource reservation system, according to some embodiments. The interface 202 may be used by a caregiver to reserve and/or manage resources for a patient (or patients). The caregiver may authenticate with the system using the interface 202.

FIG. 2C shows an example user interface 204 for a dashboard for a caregiver, according to some embodiments. The caregiver may see the dashboard after being authenticated via the user interface 202. The dashboard may include various regions. A region 210 may show providers, a region 212 may show patients, a region 214 may show scheduled appointments, a region 216 may show pending appointments, a region 218 may show appointments, a region 220 may show appointments with patients per month, a region 222 may show upcoming appointments and/or a region 224 may show appointment status per month. Any of these regions may be updated in real-time as further described below.

FIG. 2D shows an example user interface 206 for another dashboard for a caregiver, according to some embodiments. The dashboard may include various regions. A region 226 may show past appointments, a region 228 may show upcoming appointments, a region 230 may show confirmed appointments, a region 232 may show pending appointments, a region 234 may show an appointments dashboard calendar view, and/or a region 236 may show schedules. Any of these regions may be updated in real-time as further described below.

FIG. 2E shows an example user interface 208 for a dashboard for a provider, according to some embodiments. The dashboard may include various regions. A region 238 may show past appointments, a region 240 may show upcoming appointments, a region 242 may show confirmed appointments, a region 244 may show pending appointments, a region 246 may show an appointments dashboard calendar view, and/or a region 248 may show appointments referred (to another provider). Any of these regions may be updated in real-time as further described below.

FIG. 2F shows an example user interface 250 for a dashboard for a patient, according to some embodiments. The dashboard may include various regions. A region 264 may show past appointments, a region 266 may show upcoming appointments, a region 268 may show confirmed appointments, a region 270 may show pending appointments, a region 272 may show an appointments dashboard calendar view, and/or a region 274 may show appointment details. Any of these regions may be updated in real-time as further described below.

FIG. 2G shows an example user interface 252 for a calendar view for a caregiver, according to some embodiments. The view may include various regions. A region 276 may show past appointments, a region 278 may show upcoming appointments, a region 280 may show confirmed appointments, a region 282 may show pending appointments, and/or a region 284 may show a calendar with the appointments color coded (or appropriately patterned). Any of these regions may be updated in real-time as further described below.

FIG. 2H shows an example user interface 254 for a caregiver to schedule an appointment, according to some embodiments. A region 286 of the UI may be highlighted (the other region may be blurred). The region 286 may allow the caregiver to schedule appointments via the interface.

FIG. 2I shows an example user interface 256 for a patient to accept an appointment, according to some embodiments. A pop-up 288 may be shown to the patient to accept the appointment.

FIG. 2J shows an example user interface 258 for a patient to reject an appointment, according to some embodiments. A pop-up 290 may be shown to the patient to reject the appointment.

FIG. 2K shows an example user interface 260 for a caregiver to create a profile, according to some embodiments. A region 292 may show a summary of the profile (a current status, e.g., user name, location, date or time information, one or more affordances to update the profile, e.g., upload picture) and/or a region 294 that shows details of the profile. Any of these regions may be updated in real-time as further described below.

FIG. 2L shows an example user interface 262 for a dashboard for a caregiver, according to some embodiments. A region 296 may show users, a region 297 may show caregivers, a region 298 may show providers, and/or a region 299 may show list of users. Any of these regions may be updated in real-time as further described below.

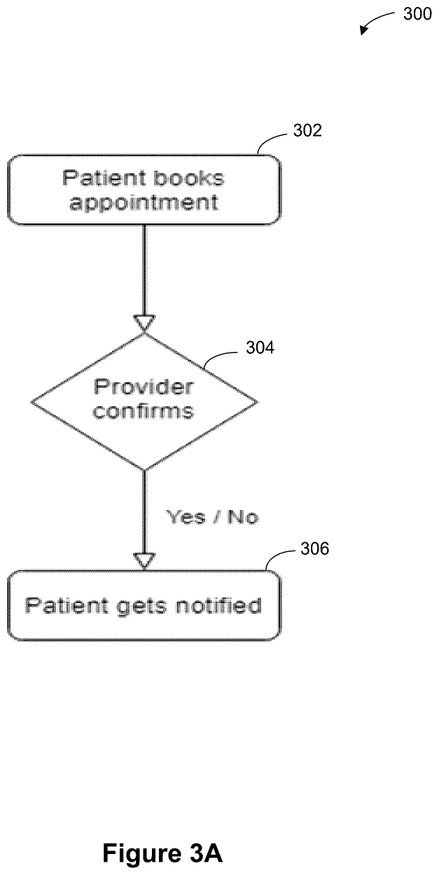

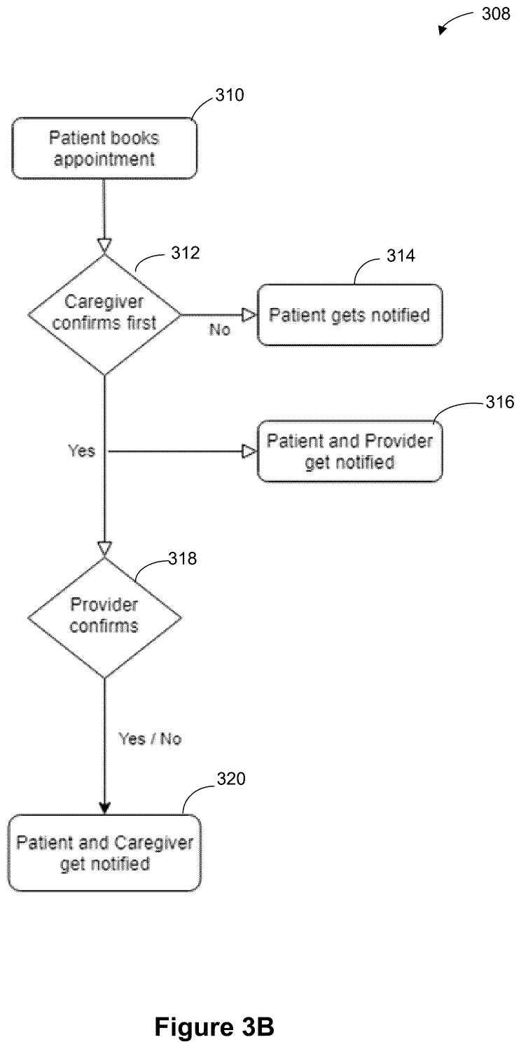

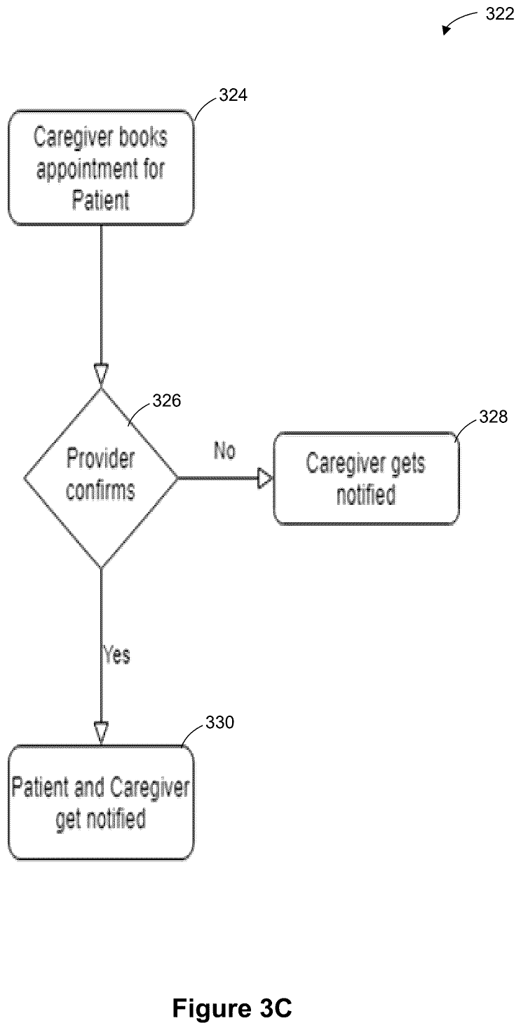

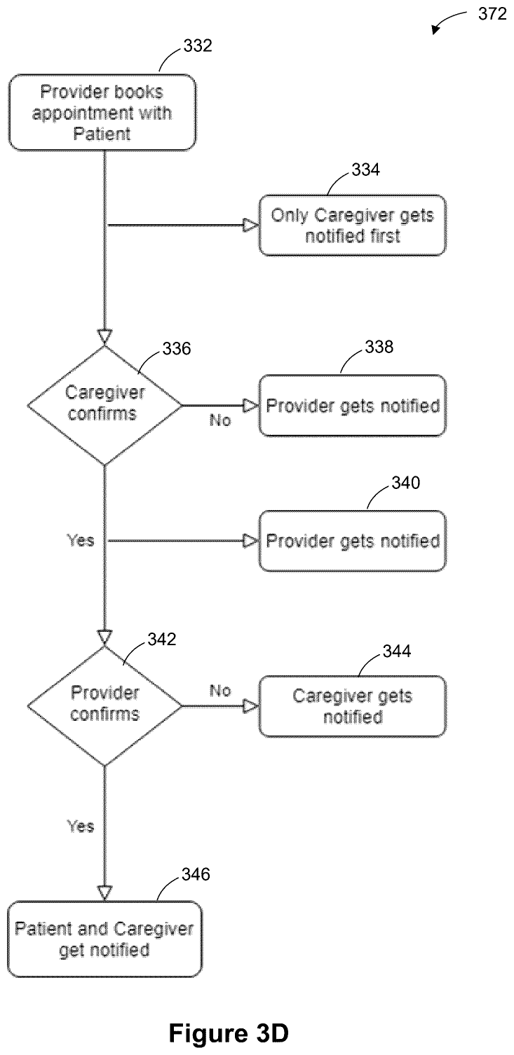

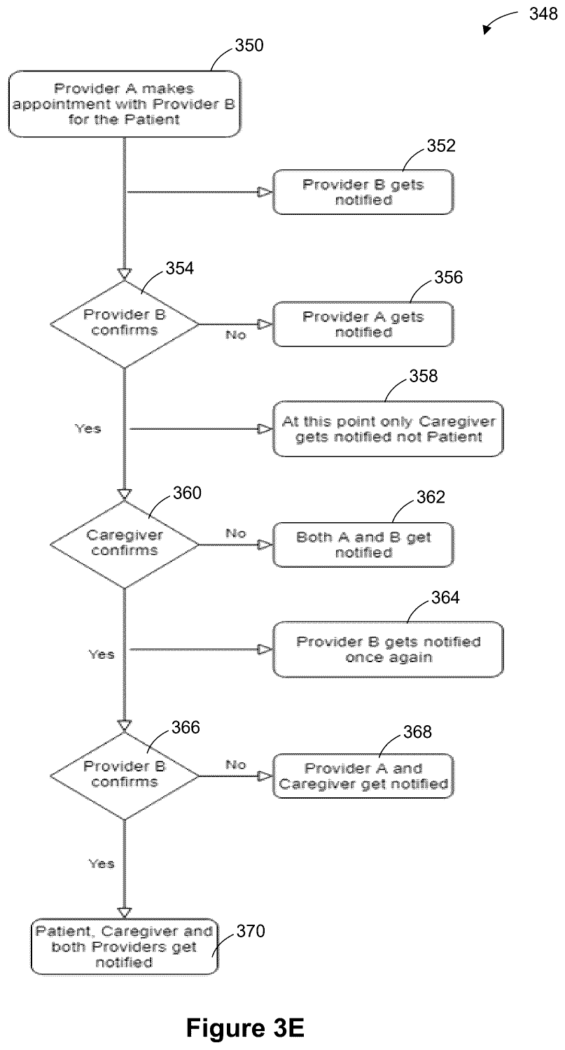

FIG. 3A shows a flowchart of an example method 300 for a patient to book an appointment with a provider without a caregiver, according to some embodiments. A patient may book (302) an appointment and a provider may confirm or reject (304). Subsequently, the patient may be notified (306). Patient does not have a caregiver in this scenario, so the patient directly (without an intermediary) books an appointment with the provider. The result may be updated in the patient's calendar (e.g., a calendar similar to the calendar view 234). Steps 302 and/or 306 may be performed via the patient UI 118, which may be similar to the caregiver UI 122, examples of which are described above in reference to FIGS. 2B, 2C, and 2D, except that the patient has information on own appointments rather than appointments for a third-party. The appointment booking and/or the provider confirmation may be performed by chain codes or smart contracts executed in the patient 110 and the provider 112.

FIG. 3B shows a flowchart of an example method 308 for a patient to book an appointment with a provider when patient has a caregiver, according to some embodiments. A patient may book (310) an appointment and a caregiver may confirm or reject (312). Subsequently, the patient may be notified (314) of the caregiver's rejection. If the caregiver confirms, the patient and the provider may be notified (316) of the caregiver's acceptance. After the caregiver confirms the appointment, the request may be reflected in both provider's and patient's calendar. After the provider confirms (318), the patient and the caregiver may be notified (320). The appointment may be reflected in both the caregiver's and patient's calendar after the provider confirms. The notifications and/or calendar updates may use the UIs described above in reference to FIGS. 1 and 2A-2E, and/or variants thereof. The appointment booking and/or the provider confirmation may be performed by chain codes or smart contracts executed in the patient 110, the provider 112, and the caregiver 114.

FIG. 3C shows a flowchart of an example method 322 for a caregiver to book an appointment with a provider on behalf of a patient, according to some embodiments. When a caregiver books (324) an appointment for a patient, the provider may confirm (326), in which case the patient and a caregiver may be notified (330); otherwise, the caregiver may be notified (328) of the provider rejecting the appointment. After a provider confirms, the acceptance may be reflected as appointments in both patient's as well as the caregiver's calendar. The notifications and/or calendar updates may use the UIs described above in reference to FIGS. 1 and 2A-2E, and/or variants thereof. The appointment booking and/or the provider confirmation may be performed by chain codes or smart contracts executed in the provider 112 and the caregiver 114.

FIG. 3D shows a flowchart of an example method 372 for a provider to book an appointment with a patient, according to some embodiments. When a provider books (332) an appointment for a patient, only the caregiver may be notified (334). After the caregiver confirms (336), the provider may get notified (340). If the caregiver declines the appointment, the provider may be notified (338) of the rejection. If the provider confirms (346), the patient and the caregiver may be notified (346). If the provider declines, only the caregiver may be notified (344). The notifications and/or calendar updates may use the UIs described above in reference to FIGS. 1 and 2A-2E, and/or variants thereof. The appointment booking and/or the provider confirmation may be performed by chain codes or smart contracts executed in the provider 112 and the caregiver 114.

FIG. 3E shows a flowchart of an example method 348 for a referral process, according to some embodiments. A provider A may make an appointment (350) with a provider B for a patient. Provider B may get notified (352). Provider B may confirm (354), in which case only caregiver may get notified (358); patient may not be notified. If provider B declines, provider A may get notified (356). Provider A may restart by referring to another provider, in that case. After provider B confirms, A may not see the appointment in their calendar; an option to display appointments by self, in the UIs described above, may enable the display. If the provider B confirms (354), the request may be forwarded to a caregiver who may confirm (360). If the caregiver declines, both providers A and B may get notified (362). If the caregiver confirms (360), the provider B may get notified (364) once again. Then provider B confirms (366) again. If the provider B declines, provider A and caregiver may be notified (368). If provider B confirms, then patient, caregiver and both providers may get notified (370).

Described herein is an example method for resource reservation using the system 100, according to some embodiments. The method may be performed at a system or a server (or a group of servers) having one or more processors, memory, one or more displays, and one or more programs stored in the memory and configured for execution by the one or more processors, the one or more programs may include instructions for performing the steps described herein. The system 100 may include a permissioned blockchain network (e.g., the network 104) coupled to one or more client devices (e.g., client devices coupled to via the application load balancer 126, client devices connected to the graphical user interfaces, client devices connected to a website or a web server). The permissioned blockchain network may include an ordering service (e.g., the orderer 108) with three orderer nodes (or an odd number of orderer nodes for optimal performance) and at least three members (e.g., the patient 110, the provider 112, and the caregiver 114). Each member may include (i) an endorsing peer node configured to endorse transactions and (ii) a committing peer node to validate and commit transactions. Each member may be configured to (i) host a respective ledger, (ii) receive, from a client device of the one or more client devices, a proposal for reserving one or more resources, (iii) in response to receiving the reservation proposal, execute a smart contract of a plurality of smart contracts on the respective ledger, to initiate a transaction for reserving the one or more resources. The system may also include a user interface component (e.g., a UI component comprising the UIs 118, 120, 122 and/or 124) coupled to the permissioned blockchain network 104. The user interface component may be configured to provide a plurality of application programmers interfaces (APIs) to access a plurality of graphical user interfaces.

In some embodiments, the at least three members may include patients (e.g., the patient 110), providers (e.g., the provider 112), and/or caregivers (e.g., the caregiver 114). Requests from the clients (e.g., patients, providers, caregivers) may be processed by their respective blockchain nodes (smart contracts) through an API call. For example, patient requests may be handled by the patient 110, provider requests may be handled by the provider 112 and caregiver requests may be handled by the caregiver 114.

In some embodiments, the plurality of smart contracts may include an appointment contract, a patient contract, a provider contract, and/or a caregiver contract. The patient contract may be executed on the patient 110, the provider contract may be executed on the provider 112, and/or the caregiver contract may be executed on the caregiver 114. The participants of the blockchain network may have the appointment contract installed on their respective blockchain nodes.

In some embodiments, the appointment contract may be configured to perform one or more operations selected from the group consisting of: creating an appointment initiated by one or more patients, one or more providers, or one or more caregivers; listing an appointment in response to queries for appointments based on a patient identifier from the respective ledger; updating one or more appointments with respect to the one or more patients, the one or more providers, or the one or more caregivers; referring appointments to let a provider refer an appointment to another provider; and querying all the appointments based on a query pattern.

In some embodiments, the patient contract may be configured to perform one or more operations selected from the group consisting of: enrolling a new patient into the permissioned blockchain network 104; querying patient information in the respective ledger, in response to a query from one or more patients, one or more providers, or one or more caregivers; and updating patient information in response to a request from the one or more patients.

In some embodiments, the provider contract may be configured to perform one or more operations selected from the group consisting of: enrolling a new provider into the permissioned blockchain network 104; querying provider information in the respective ledger, in response to a query from one or more patients, one or more providers, or one or more caregivers; and updating provider information in response to a request from the one or more providers.

In some embodiments, the caregiver contract may be configured to perform operations selected from the group consisting of: enrolling a new caregiver into the permissioned blockchain network 104; querying caregiver information in the respective ledger, in response to a query from one or more patients, one or more providers, or one or more caregivers; updating caregiver information in response to a request from the one or more caregivers; and controlling appointment of a patient's appointment.

In some embodiments, the plurality of graphical user interfaces may include one or more graphical user interfaces for client login, client signup, a plurality of dashboards, each dashboard corresponding to a respective member type, an interface for a calendar view, an interface for booking resources, an interface for accepting resource reservation, an interface for rejecting resource booking, an interface for client profile management, an interface for displaying current users. Examples of UIs are described above in reference to FIGS. 1 and 2A-2E, according to some embodiments.

In some embodiments, the plurality of graphical user interfaces may include a corresponding graphical user interface for patients (e.g., the UI 118), a corresponding interface for providers (e.g., the UI 120), and a corresponding interface for caregivers (e.g., the UI 122).

In some embodiments, the plurality of graphical user interfaces may include an interface with one or more affordances for providers to refer an appointment to another provider (an example of which was described above in reference to FIG. 2E).

In some embodiments, the permissioned blockchain network 104 may be configured to, at a member of the at least three members, receive a connection request from a client device of the one or more client devices. The permissioned blockchain network 104 may also be configured to, at a smart contract: process the connection request to select (i) a graphical user interface of the plurality of graphical user interfaces based on the client (e.g., the patient UI 118 may be shown for a connection from a patient, the provider UI 120 may be shown for a connection request from a patient, a caregiver UI 122 may be shown for a connection request from a caregiver) and/or (ii) an API (e.g., the API 116) corresponding to the graphical user interface; provide, via the API, the graphical user interface, to the client; receive, via the graphical user interface, a selection of a resource of a plurality of resources (e.g., a calendar of available appointment dates may be shown and a user may select a particular date, a collection of providers may be shown and a user may select a particular provider, a selection of other providers may be shown and a provider may select a provider to refer a patient, a collection of medical or computing resources may be shown and a user may select a resource to reserve, and so on), from the client device; and in response to the selection of the resource, perform, using the plurality of graphical user interfaces and/or the respective ledger, one or more operations selected from the group consisting of: client login, client signup, and display (i) one or more dashboards, each dashboard corresponding to a respective member type, (ii) an interface for a calendar view, (ii) an interface for booking resources, (iii) an interface for accepting resource reservation, (iv) an interface for rejecting resource booking, (v) an interface for client profile management, or (vi) an interface for displaying current users.

In some embodiments, the application load balancer may be targeted towards one or more APIs. An API may determine which smart contract needs to execute based on the request that is received from a graphical user interface (e.g., a request triggered via a selection from a user interface or screen on a client device, via an application). A backend API may handle this logic. Requests received from a client device may be processed by one or more graphical user interfaces, the application load balancer and/or APIs.

While embodiments and alternatives have been disclosed and discussed, the invention herein is not limited to the particular disclosed embodiments or alternatives but encompasses the full breadth and scope of the invention including equivalents, and the invention is not limited except as set forth in and encompassed by the full breadth and scope of the claims herein.

What is claimed is:

1. A system for resource reservation using smart contracts, the system comprising:
a permissioned blockchain network coupled to one or more client devices, wherein the permissioned blockchain network includes an ordering service and at least three members, wherein each member is configured to (i) host a respective ledger, (ii) receive, from a client device of the one or more client devices, a proposal for reserving one or more resources, (iii) in response to receiving the reservation proposal, execute a smart contract of a plurality of smart contracts on the respective ledger, to initiate a transaction for reserving the one or more resources; and
a user interface component coupled to the permissioned blockchain network, wherein the user interface component is configured to provide a plurality of application programmers interfaces (APIs) to access a plurality of graphical user interfaces.

2. The system of claim 1, wherein the at least three members includes patients, providers, and caregivers.

3. The system of claim 1, wherein the plurality of smart contracts comprises an appointment contract, a patient contract, a provider contract and a caregiver contract.

4. The system of claim 3, wherein the appointment contract is configured to:
create an appointment initiated by one or more patients, one or more providers, or one or more caregivers;
list an appointment in response to queries for appointments based on a patient identifier from the respective ledger;
update one or more appointments with respect to the one or more patients, the one or more providers, or the one or more caregivers;
refers appointments to let a provider refer an appointment to another provider; and
query all the appointments based on a query pattern.

5. The system of claim 3, wherein the patient contract is configured to:
enroll a new patient into the permissioned blockchain network;
query patient information in the respective ledger, in response to a query from one or more patients, one or more providers, or one or more caregivers; and
update patient information in response to a request from the one or more patients.

6. The system of claim 3, wherein the provider contract is configured to:
enroll a new provider into the permissioned blockchain network;
query provider information in the respective ledger, in response to a query from one or more patients, one or more providers, or one or more caregivers; and
update provider information in response to a request from the one or more providers.

7. The system of claim 3, wherein the caregiver contract is configured to:
enroll a new caregiver into the permissioned blockchain network;
query caregiver information in the respective ledger, in response to a query from one or more patients, one or more providers, or one or more caregivers;
update caregiver information in response to a request from the one or more caregivers; and
control appointment of a patient's appointment.

8. The system of claim 1, wherein the plurality of graphical user interfaces includes one or more graphical user interfaces for client login, client signup, a plurality of dashboards, each dashboard corresponding to a respective member type, an interface for a calendar view, an interface for booking resources, an interface for accepting resource reservation, an interface for rejecting resource booking, an interface for client profile management, an interface for displaying current users.

9. The system of claim 1, wherein the plurality of graphical user interfaces includes a corresponding graphical user interface for patients, a corresponding interface for providers, and a corresponding interface for caregivers.

10. The system of claim 1, wherein the plurality of graphical user interfaces includes an interface with one or more affordances for providers to refer an appointment to another provider.

11. The system of claim 1, wherein the permissioned blockchain network is configured to:
at a member of the at least three members:
receive a connection request from a client device of the one or more client devices;
at a smart contract:
process the connection request to select (i) a graphical user interface of the plurality of graphical user interfaces based on the client device and (ii) an API corresponding to the graphical user interface;
provide, via the API, the graphical user interface, to the client device;
receive, via the graphical user interface, a selection of a resource of a plurality of resources, from the client device; and
in response to the selection of the resource, perform, using the plurality of graphical user interfaces and/or the respective ledger, one or more operations selected from the group consisting of: client login, client signup, and display (i) one or more dashboards, each dashboard corresponding to a respective member type, (ii) an interface for a calendar view, (ii) an interface for booking resources, (iii) an interface for accepting resource reservation, (iv) an interface for rejecting resource booking, (v) an interface for client profile management, or (vi) an interface for displaying current users.

12. The system of claim 1, wherein each member includes (i) an endorsing peer node configured to endorse transactions and (ii) a committing peer node to validate and commit transactions.

13. A method for resource reservation using smart contracts, the method comprising:
at a permissioned blockchain network coupled to one or more client devices, wherein the permissioned blockchain network includes an ordering service with three orderer nodes and at least three members:
at each member (i) hosting a respective ledger, (ii) receiving, from the one or more client devices, using one or more application programmers interfaces (APIs), via one or more graphical user interfaces of a plurality of graphical user interfaces, a proposal for reserving one or more resources, (iii) in response to receiving the reservation proposal, (a) executing a smart contract of a plurality of smart contracts on the respective ledger, to initiate a transaction for reserving the one or more resources, and (b) indicating, to a client device, a successful reservation or a rejection of the reservation of one or more resources, via the one or more graphical user interfaces.

14. The method of claim 13, wherein the at least three members includes patients, providers, and caregivers.

15. The method of claim 13, wherein the plurality of smart contracts comprises an appointment contract, a patient contract, a provider contract and a caregiver contract.

16. The method of claim 15, wherein the appointment contract comprises operations selected from the group consisting of:
creating an appointment initiated by one or more patients, one or more providers, or one or more caregivers;
listing an appointment in response to queries for appointments based on a patient identifier from the respective ledger;
updating one or more appointments with respect to one or more patients, one or more providers, or one or more caregivers;
referring appointments to let a provider refer an appointment to another provider; and
querying all the appointments based on a query pattern.

17. The method of claim 15, wherein the patient contract comprises operations selected from the group consisting of:
enrolling a new patient into the permissioned blockchain network;
querying patient information in the respective ledger, in response to a query from one or more patients, one or more providers, or one or more caregivers; and
updating patient information in response to a request from the one or more patients.

18. The method of claim 15, wherein the provider contract comprises operations selected from the group consisting of:
enrolling a new provider into the permissioned blockchain network;
querying provider information in the respective ledger, in response to a query from one or more patients, one or more providers, or one or more caregivers; and
updating provider information in response to a request from the one or more providers.

19. The method of claim 15, wherein the caregiver contract comprises operations selected from the group consisting of:
enrolling a new caregiver into the permissioned blockchain network;
querying caregiver information in the respective ledger, in response to a query from one or more patients, one or more providers, or one or more caregivers;
updating caregiver information in response to a request from the one or more caregivers; and
controlling appointment of a patient's appointment.

20. The method of claim 13, comprising:
at a member of the at least three members:
receiving a connection request from a client device of the one or more client devices;
at a smart contract:
processing the connection request to select (i) a graphical user interface of the plurality of graphical user interfaces based on the client device and (ii) an API corresponding to the graphical user interface;
providing, via the API, the graphical user interface, to the client device;
receiving, via the graphical user interface, a selection of a resource of a plurality of resources, from the client device; and
in response to the selection of the resource, performing, using the plurality of graphical user interfaces and/or the respective ledger, one or more operations selected from the group consisting of: client login, client signup, and display (i) one or more dashboards, each dashboard corresponding to a respective member type, (ii) an interface for a calendar view, (ii) an interface for booking resources, (iii) an interface for accepting resource reservation, (iv) an interface for rejecting resource booking, (v) an interface for client profile management, or (vi) an interface for displaying current users.
